(19) United States
(12) Patent Application Publication
(10) Pub. No.: US 2013/0137097 A1
(43) Pub. Date: May 30, 2013

(54) HIGH THROUGHPUT SINGLE NUCLEOTIDE POLYMORPHISM ASSAY

(71) Applicant: AGRIGENETICS, INC., Indianapolis, IN (US)

(72) Inventors: Wenxiang Gao, Carmel, IN (US); Siva P. Kumpatla, Carmel, IN (US); Robert Martin Benson, Ellsworth, WI (US); James Todd Gerdes, Ontario (CA)

(73) Assignee: AGRIGENETICS, INC., Indianapolis, IN (US)

(21) Appl. No.: 13/685,903

(22) Filed: Nov. 27, 2012

Related U.S. Application Data
(60) Provisional application No. 61/564,464, filed on Nov. 29, 2011.

(51) Int. Cl. C12Q 1/68

(52) U.S. Cl. CPC C12Q 1/6827
USPC 435/6.11

(57) ABSTRACT

A method consisting of a homogeneous assay detection system for a PCR process using FRET for detection and zygosity analysis of the HaAHASL1-A122(At)T single nucleotide polymorphism in sunflower is provided. The method provides specific sunflower-genome primers that can be used to detect the presence or absence of the HaAHASL1-A122(At)T single nucleotide polymorphism. The primer combinations for use in an endpoint PCR assay capable of determining zygosity and for assisting in breeding introgression are described.

### CROSS-REFERENCE TO RELATED APPLICATIONS

This Application claims the benefit of U.S. Provisional Application 61/564,464, filed on Nov. 29, 2011, which is expressly incorporated by reference herein.

### REFERENCE TO SEQUENCE LISTING SUBMITTED ELECTRONICALLY

The official copy of the sequence listing is submitted electronically via EFS-Web as an ASCII formatted sequence listing with a file named “69826USNP_SEQ_ID_ST25”, created on Nov. 27, 2012, and having a size of 2 kb and is filed concurrently with the specification. The sequence listing contained in this ASCII formatted document is part of the specification and is herein incorporated by reference in its entirety.

### BACKGROUND DISCLOSURE

The subject disclosure concerns a method consisting of a homogeneous assay detection system for a PCR process using FRET to detect the AHASL1-A122(AT)T single nucleotide polymorphism in Helianthus annuus L. The AHASL1-A122(AT)T allele imparts tolerance to imidazolinone herbicides. The detection method can be used for breeding introgression of the AHASL1-A122(AT)T allele, thereby imparting herbicide tolerance into lines of Helianthus annuus. The subject disclosure is applicable for agricultural biotechnology.

Herbicide tolerant trait usage within crop management systems provides growers more efficient and profitable crop management solutions. Crop management systems which deploy the use of herbicide tolerant traits increase the use of double-crop and no-till cropping systems, improve weed management techniques, reduce energy costs, and generally provide cost reduction as compared to conventional crop management systems. Once an herbicide tolerant trait is identified, the development of methods which can be use to reliably detect the trait are essential for the breeding introgression of the herbicide tolerant trait into crops.

An herbicide tolerant trait which imparts tolerance to several classes of herbicides including imidazolinones has been identified. see Kolkman et al. (2004) and WO2007/005581. A single nucleotide polymorphism (SNP) mutation of the HaAHASL1 coding sequence was characterized to provide tolerance for imidazolinone herbicides within chromosome 9 of Helianthus annuus L. Unfortunately, high-throughput detection of the HaAHASL1-A122(At)T specific mutation is difficult due to the presence of paralogous sequences which share high levels of sequence similarity with HaAHASL1. Existing assays used to detect the HaAHASL1-A122(At)T mutation, in particular gel electrophoresis based assays, are low throughput, inconvenient, time-consuming, and expensive. To detect this imidazolinone tolerant allele, a high-throughput, cost effective and efficient genotyping assay is desirable.

The development of a single nucleotide polymorphism (SNP) assay for the detection of the HaAHASL1-A122(At)T allele in sunflower is described herein. The assay provides an effective breeding introgression method for marker assisted selection (MAS) to support imidazolinone-tolerance trait breeding introgression into sunflower lines, thereby significantly increasing breeding selection efficiency. The assay reduces the cost and time to synthesize a new assay relative to other quantitative PCR technologies. Moreover, the assay is successful where other quantitative or detection technologies have been tried and failed.

### BRIEF SUMMARY

The subject disclosure provides a method consisting of a homogeneous assay detection system for a PCR process using FRET for detecting the presence or absence of a HaAHASL1-A122(At)T SNP within the HaAHASL1 gene, comprising:

isolating a genomic DNA sample from Helianthus annuus;

adding a set of oligonucleotide primers to said isolated genomic DNA sample, wherein said set of oligonucleotide primers are comprised of a mutant allele detection common primer consisting of SEQ ID NO:3, a wildtype allele detection common primer consisting of SEQ ID NO:4, a downstream common primer consisting of SEQ ID NO:5, and fluorescent-labeled primers;

subjecting said isolated genomic DNA sample and said set of oligonucleotide primers to an amplification process; and,

detecting at least one amplified product, wherein the amplified product indicates the presence or absence of a HaAHASL1-A122(At)T SNP.

One embodiment of the disclosure concerns a method, wherein said amplified product consists of 84 base pairs.

Another embodiment of the disclosure concerns a method wherein the site of said present or absent SNP is located between SEQ ID NO:3 and SEQ ID NO:5 of Helianthus annuus chromosome 9.

Another embodiment of the disclosure concerns a method wherein the site of said present or absent SNP is located between SEQ ID NO:4 and SEQ ID NO:5 of Helianthus annuus chromosome 9.

Another embodiment of the disclosure concerns identifying the presence or absence of the HaAHASL1-A122(At)T SNP in different plant lines using the method consisting of a homogeneous assay detection system for a PCR process using FRET. Thus, another embodiment of the subject disclosure describes a method consisting of a homogeneous assay detection system for a PCR process using FRET that can be used to identify plant lines which contain the HaAHASL1-A122(At)T SNP.

Another embodiment of the disclosure concerns the identification of the presence or absence of the HaAHASL1-A122(At)T SNP in progeny plants using the method consisting of a homogeneous assay detection system for a PCR process using FRET. A parent plant comprising the HaAHASL1-A122(At)T SNP is crossed with a second plant line in an effort to impart one or more additional traits of interest in the progeny. The method consisting of a homogeneous assay detection system for a PCR process using FRET can be utilized to screen for the presence or absence of the HaAHASL1-A122(At)T SNP in the resulting progeny plants.

Another embodiment of the disclosure concerns the development of molecular marker systems which can be used for marker assisted breeding introgression. Such molecular marker systems can be used to accelerate breeding introgression strategies and to establish linkage data. The method consisting of a homogeneous assay detection system for a PCR process using FRET can be utilized to screen for the presence or absence of the HaAHASL1-A122(At)T SNP as a molecular marker system which can be used for marker assisted breeding introgression.

Another embodiment of the disclosure concerns the application of the method consisting of a homogeneous assay detection system for a PCR process using FRET for determining zygosity, wherein the HaAHASL1-A122(At)T SNP is determined to be present as homozygous within the genome of Helianthus annuus, or wherein the HaAHASL1-A122(At)T SNP is determined to be present as hemizygous within the genome of Helianthus annuus, or wherein the HaAHASL1-A122(At)T SNP is determined to not be present (null) within the genome of Helianthus annuus.

Another embodiment the disclosure concerns identifying imidazolinone herbicide tolerance resulting from the presence of the HaAHASL1-A122(At)T SNP in different plant lines using the method consisting of a homogeneous assay detection system for a PCR process using FRET. Thus, an embodiment of the subject disclosure describes a method consisting of a homogeneous assay detection system for a PCR process using FRET that can be used to identify plant lines which are tolerant to imidazolinone herbicides.

Another embodiment of the disclosure concerns the application of the method consisting of a homogeneous assay detection system for a PCR process using FRET for confirming the presence of the HaAHASL1-A122(At)T SNP in a stack of transgenes, wherein additional transgenes are introgressed into a plant containing the HaAHASL1-A122(At)T SNP via traditional plant breeding introgression or through transformation of a second transgene within the genome of a plant.

### BRIEF DESCRIPTION OF THE FIGURE

FIG. 1 depicts the results of a method consisting of a homogeneous assay detection system for a PCR process using FRET for HaAHASL1 genotyping and detection of the single nucleotide polymorphism, HaAHASL1-A122(At)T. Each genotype was replicated six times. For each sample, fluorescent emission reads were plotted with the X-axis representing the FAM intensity and Y-axis representing the JOE intensity. Four clusters were identified on the graph. Upper Left hand corner=homozygous wildtype sunflower plants; Lower Right Hand corner=homozygous mutant sunflower plants containing the HaAHASL1-A122(At)T) allele (a single nucleotide polymorphism; Upper Right hand corner=Hemizygous control; and Lower Left Hand corner=null experimental control.

### BRIEF DESCRIPTION OF THE SEQUENCES

SEQ ID NO:1 is the 86 base pair fragment of wildtype DNA which is amplified in the method consisting of a homogeneous assay detection system for a PCR process using FRET for detection of the non-mutant allele of HaAHASL1 sequence.

SEQ ID NO:2 is the 84 base pair fragment of DNA containing the mutant allele which is amplified in the method consisting of a homogeneous assay detection system for a PCR process using FRET for detection of the single nucleotide polymorphism of HaAHASL1-A122(At)T

SEQ ID NO:3 is the mutant allele detection common primer, 043-0001.1.A1 containing a tail on the 5′ end that binds to a KBiosciences reaction kit primer which is labeled with the fluorescent dye, 5-carboxyfluorescein (FAM). This primer was specifically designed to amplify the HaAHASL1-A122(At)T allele.

SEQ ID NO:4 is the wildtype allele detection common primer, 043-0001.2.A2 containing a tail on the 5′ end that binds to a KBiosciences reaction kit primer which is labeled with the fluorescent dye, 2′,7′-dimethoxy-4′,5′-dichloro-6-carboxyfluorescein (JOE). This primer was designed to specifically amplify the wildtype HaAHASL1 allele.

SEQ ID NO:5 is the downstream common primer, 043.0001.8.0 which hybridizes to a sequence downstream of the HaAHASL1-A122(At)T mutation site and was used to amplify both the mutant and wildtype alleles

### DETAILED DESCRIPTION

The subject disclosure provides a method consisting of a homogeneous assay detection system for a PCR process using FRET for determining the zygosity of a trait within plants. More specifically, the present disclosure relates in part to a method consisting of a homogeneous assay detection system for a PCR process using FRET for detecting the presence of the HaAHASL1-A122(At)T single nucleotide polymorphism in Helianthus annuus.

The homogeneous assay detection system for a PCR process is a genotyping system which deploys the use of Fluorescent Resonance Energy Transfer (FRET) for the detection of the presence or absence of a SNP. This genotyping system utilizes a common oligonucleotide primer to initiate the PCR process. This common primer is tailed with a DNA sequence at the 5′ portion of the primer and the tail is not directed to the amplicon region of interest; as such this tail is essentially inert. The 3′ portion of the primer is directed to the amplicon region of interest and therefore drives the specificity of the reaction. A downstream common primer is included in the reaction. The downstream common primer and common primer are used to amplify a specific DNA fragment. Also included in the PCR reaction is a single fluorescent-labeled oligonucleotide primer that is identical in sequence to the tail region of the common primer. Finally, included in the reaction is a 3′ quencher labeled oligonucleotide primer which is designed antisense to the fluorescent-labeled oligonucleotide.

Due to the complementarity of the two-labeled oligonucleotides (one fluorescent-labeled oligonucleotide primer and a second quencher labeled oligonucleotide primer) they hybridize to each other. This hybridization brings the quencher label in very close proximity to the fluorescent label (which is also described as a fluorophore), thereby rendering all fluorescent signal from the fluorophore molecule quenched when excited at the optimal excitation wavelength of the fluorophore.

A PCR process is initiated and PCR products are generated using common primers. After the first few cycles of PCR the antisense sequence to the fluorescent-labeled primer is generated. The fluorescent-labeled PCR primer is then able to initiate synthesis during the PCR, and does so. This produces an amplicon or PCR generated DNA fragment containing the fluorophore molecule. With the synthesis of this DNA fragment, the quenching oligonucleotide is no longer able to hybridize to the fluorescently-labeled oligonucleotide as the PCR process produces double stranded amplicon DNA. As the quenching oligonucleotide can no longer hybridize to the fluorescently-labeled oligonucleotide, a fluorescent signal is generated which is directly proportional to the amount of PCR product generated.

Variations of this genotyping system can be used for end point and real timer analysis to detect an allele specific SNP. This method utilizes the same fluorophore/quencher oligonucleotide primer pair in conjunction with a common downstream oligonucleotide primer and a common oligonucleotide primer, as described above. The reaction scheme is identical, except for a few modifications.

Completion of the allele specific SNP genotyping requires the use of two fluorescent-labeled primers and corresponding quencher oligonucleotides. Each primer is tailed with a unique sequence, to which in the reaction is included a unique 5′ fluorescent labeled primer. While many dyes are considered appropriate, two suitable dyes are FAM and JOE, both derivatives of fluorescein but spectrally resolvable from each other. Two common primers (one designed for the mutant allele and the second designed for the wildtype allele) are designed to hybridize to the DNA of interest which is the non-tailed portion of the primer. The common primers also contain a tailed portion of sequence which is identical in sequence to one of the two fluorescent-labeled oligonucleotides. In the non-tailed portion of the primer the two common primers typically differ only by a single nucleotide at their 3′ terminal base. Each primer is directed to the single nucleotide polymorphic base in the DNA of interest. PCR is conducted and the two primers only initiate DNA synthesis when the 3′ base is perfectly matched. When a mismatch occurs DNA synthesis does not proceed.

During the PCR reaction only one specific common primer is able to initiate DNA synthesis for the genotype that contains homozygous mutant alleles or for the genotype that contains homozygous wildtype alleles. In the case of a genotype that is heterozygous for both wildtype and mutant alleles, both common primers are able to initiate DNA synthesis. The resulting PCR reactions incorporate the tailed portion of the common primer into the PCR product. After the first few cycles of PCR the antisense sequence to the fluorescent-labeled primer is generated. The fluorescent-labeled PCR primer is then able to initiate synthesis during the PCR, and does so. This produces an amplicon or PCR generated DNA fragment containing the fluorophore molecule. With the synthesis of this DNA fragment, the quenching oligonucleotide is no longer able to hybridize to the fluorescent-labeled oligonucleotide as the PCR process produces double stranded amplicon DNA. As the quenching oligonucleotide can no longer hybridize to the fluorescent-labeled oligonucleotide, a fluorescent signal is generated according to which of the common oligonucleotides has initiated the synthesis. The reaction is then read on a fluorescent plate reader for both fluorophores. The resulting data is then plotted and a cluster plot of one fluorophore (e.g. FAM) over the other fluorophore (e.g. JOE) is generated. The resulting genotypes are then able to be determined based on the cluster plots.

The assay results are based on a plus/minus strategy, by which a “plus” signifies the sample is positive for the assayed gene and a “minus” signifies the sample is negative for the assayed gene. These assays typically utilize two allele specific common primers and a common primer for identifying the HaAHASL1-A122(At)T single nucleotide polymorphism (mutant allele) and the wild-type HaAHASL1 (wildtype allele) in the same PCR reaction. The application of this endpoint assay allows for the use of the method consisting of a homogeneous assay detection system for a PCR process using FRET for determination of zygosity.

Specific detection of PCR products is the most robust method for ensuring the accurate monitoring of a presence of DNA region of interest and detection of single nucleotide polymorphism. Other known assays, such as hydrolysis probe methods, are widely used. However these types of methods are expensive to perform, due to the requirement for double labeled probes. Additional assays in particular gel electrophoresis based assays are known in the art. Unfortunately, these methods are low throughput, inconvenient, time-consuming, and expensive. A high-throughput, cost effective and efficient genotyping assay such as the method consisting of a homogeneous assay detection system for a PCR process using FRET is desirable. In addition this single nucleotide polymorphism detection method should provide robust detection in the presence of paralogous sequences which share high levels of sequence similarity. The single nucleotide polymorphism assay of the subject disclosure provides an effective method for the accurate monitoring of a presence of DNA region of interest and detection of single nucleotide polymorphism.

“Oligonucleotide primers” are isolated polynucleotide sequences that anneal to a complementary target DNA strand by nucleic acid hybridization to form a hybrid between the primer and the target DNA strand, and can be used in conjunction with a polymerase, e.g., a DNA polymerase. Oligonucleotide primers can be described as oligonucleotides or primers. The oligonucleotide primers of the present disclosure refer to their use for amplification of a target nucleic acid sequence (also described as an amplicon), e.g., by the polymerase chain reaction (PCR) or other conventional nucleic-acid amplification processes. In a preferred embodiment a method consisting of a homogeneous assay detection system for a PCR process using FRET is used to amplify a target nucleic acid sequence (also described as an amplicon) using oligonucleotide primers.

Oligonucleotide primers are generally 12-50 base pairs or more in length. Such primers hybridize specifically to a target sequence under high stringency hybridization conditions. Preferably, primers according to the present disclosure have complete sequence similarity with the target sequence, although primers differing from the target sequence and that retain the ability to hybridize to target sequences may be designed by conventional methods. Other modifications may be introduced to incorporate degenerate sequences to either the 5′ or 3′ end of the primer. The addition of a degenerate sequence which is described as a “tail” may be included to bind to a single fluorescent-labeled oligonucleotide primer.

Specific oligonucleotide primers were designed comprising a fluorescent reporter (fluorophore) or a quencher. The fluorophore molecule is added to an oligonucleotide primer during the synthesis of the oligonucelotide primer thereby labeling the oligonucleotide primer. Likewise, other molecules can be added to oligonucleotide primers during synthesis, such as a quencher molecule. The addition of these molecules to an oligonucleotide primer does not impair the function of the oligonucleotide primer when hybridizing to single stranded DNA and producing a new strand of DNA via an amplification process.

Numerous fluorophores have been developed that excite at specific wavelengths and are known in the art. Excitation of the fluorophore results in the release of a fluorescent signal by the fluorophore which can be quenched by a quencher located in close proximity to the fluorophore. When the quencher is disassociated from the fluorophore, the fluorescent signal is no longer quenched and accumulation of the fluorescent signal, which is directly correlated with the amount of target DNA, can be detected in real-time or as an end-point reaction with an automated fluorometer. The fluorophores may be used in combination, wherein the excitation and emission spectra are significantly different as to allow multiple detection of two or more fluorophores. Some fluorophores useful in the subject methods include: a HEX fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, or a ROX fluorescent dye. A fluorophore for use with the method consisting of a homogeneous assay detection system for a PCR process using FRET of the subject disclosure includes a FAM fluorescent dye or a JOE fluorescent dye.

Quenchers have been developed to quench fluorophores at a specific wavelength and are known in the art. When the quencher is located in close approximation to the fluorophore, the fluorophore transfers energy to the quencher. The quencher dissipates this energy and returns to a native ground state through nonradiative decay. In nonradiative or dark decay, the energy transferred from the fluorophore is given off as molecular vibrations. Selection of a quencher considers qualities such as low background fluorescence, high sensitivity, and maximal spectral overlap to provide a quencher that can enable a wider use of fluorophores. Some quenchers useful in the subject methods include: Dabcyl quenchers, Tamra quenchers, Qxl quencher, Iowa black FQ quencher, Iowa black RQ quencher, or an IR Dye QC-1 quencher. An example of a quencher would include an Blackhole quencher labeled on an oligonucleotide primer which is designed antisense to the FAM or JOE labeled oligonucleotide.

Single nucleotide polymorphisms within genes encoding the AHAS enzyme have been identified and characterized and are known to provide tolerance to herbicides. Mutations of the HaAHASL1 coding sequence which impart tolerance for imidazolinone herbicides have been identified in Helianthus annuus L. see Kolkman et al. (2004) and WO2007/005581. A single nucleotide polymorphism (SNP) mutation of the HaAHASL1 coding sequence, wherein the native alanine at position 122 is mutated to a threonine via a single nucleotide polymorphism (characterized as a guanine to adenosine transition), was characterized to provide tolerance for imidazolinone herbicides within Helianthus annuus L.

The single nucleotide polymorphism which encodes resistance to AHAS-inhibiting herbicides in sunflower has been identified and introgressed into elite inbred lines for the purpose of developing and deploying herbicide resistant cultivars and hybrids. The identification of sunflower lines which provide tolerance to AHAS-inhibiting herbicides such as imidazolinone provides sunflower producers new cropping systems for the control of broadleaf weeds. Sunflower lines marketed as CLHA-Plus CLEARFIELD®, are imidazolinone tolerant and known to carry the HaAHASL1-A122(At)T mutant allele. A preferred embodiment of the disclosure is a method consisting of a homogeneous assay detection system for a PCR process using FRET which can be used to detect the presence or absence of the HaAHASL1-A122(At)T in sunflower plants.

The genomic sequence of the HaAHASL1 wildtype allele is provided as SEQ ID NO:1. The genomic sequence containing the HaAHASL1-A122(At)T mutant single nucleotide polymorphism allele is provided as SEQ ID NO:2. The location of the single nucleotide polymorphism which results in imidazolinone tolerance is located at base pair 65 of SEQ ID NO:1 and base pair 65 of SEQ ID NO:2.

Based on these genomic sequences, specific common primers were generated. A method consisting of a homogeneous assay detection system for a PCR process using FRET of the subject disclosure demonstrated that the HaAHASL1-A122(At)T mutant single nucleotide polymorphism allele can be identified in different sunflower lines with these specific common primer sets. The method consisting of a homogeneous assay detection system for a PCR process using FRET can be used to uniquely identify the presence or absence of the HaAHASL1-A122(At)T single nucleotide polymorphism in sunflower lines.

In one embodiment, the HaAHASL1-A122(At)T method consisting of a homogeneous assay detection system for a PCR process using FRET amplifies an 84 by fragment. A HaAHASL1-A122(At)T specific common primer binds to the single nucleotide polymorphism mutant allele. A HaAHASL1 specific common primer binds to the wildtype allele. A downstream common primer binds to a sequence downstream of the HaAHASL1A122(At)T mutation site and is used to amplify both the mutant and wildtype alleles. The primers used for the amplification of the HaAHASL1-A122(At)T single nucleotide polymorphism were tested for PCR efficiencies. Primer combinations and PCR amplification conditions were developed for multiplexing capabilities to produce an endpoint zygosity assay.

Detection techniques of the subject disclosure can be used in conjunction with plant breeding introgression to determine which progeny plants contain a single nucleotide polymorphism after a parent plant containing a single nucleotide polymorphism is crossed with another plant line in an effort to impart one or more additional traits of interest in the progeny.

The subject method consisting of a homogeneous assay detection system for a PCR process using FRET is useful in, for example, sunflower breeding introgression programs as well as quality control, especially for commercial production of sunflower seeds. This method can also benefit product registration and product stewardship. This method can be used for accelerated breeding introgression strategies. The detection techniques of the subject disclosure are especially useful in conjunction with plant breeding introgression, to determine which progeny plants comprise the single nucleotide polymorphism, after a parent plant containing a single nucleotide polymorphism is crossed with another plant line in an effort to impart the single nucleotide polymorphism into the progeny. The disclosed method consisting of a homogeneous assay detection system for a PCR process using FRET benefits sunflower breeding introgression programs as well as quality control, especially for commercialized sunflower seeds.

This disclosure further includes the processes of making sunflower plant crosses and using methods of the subject disclosure. For example, the subject disclosure includes a method for producing a progeny seed by crossing a plant containing a single nucleotide polymorphism with a second and genetically different plant (e.g. in-bred parent which does not contain the SNP), harvesting the resultant progeny seed, and detecting for a single nucleotide polymorphism using the method consisting of a homogeneous assay detection system for a PCR process using FRET.

A herbicide-tolerant sunflower plant can be bred by first sexually crossing a first parental sunflower plant consisting of a sunflower plant grown from seed of a line containing the single nucleotide polymorphism, and a second parental sunflower plant, thereby producing a plurality of first progeny plants; and then selecting a first progeny plant that contains the single nucleotide polymorphism and is resultantly resistant to a herbicide; and selfing the first progeny plant, thereby producing a plurality of second progeny plants; and then selecting from the second progeny plants a plant that contain the single nucleotide polymorphism and is resultantly resistant to a herbicide.

These steps can further include the back-crossing of the first progeny plant or the second progeny plant to the second parental sunflower plant or a third parental sunflower plant. A sunflower crop comprising sunflower seeds which contain the single nucleotide polymorphism, or progeny thereof, can be rapidly detected using the method consisting of a homogeneous assay detection system for a PCR process using FRET and then be planted. The method consisting of a homogeneous assay detection system for a PCR process using FRET can improve the efficiency of this process.

The present disclosure can be used for a marker assisted breeding (MAB) method. The present disclosure can be used in combination with other methods (such as, AFLP markers, RFLP markers, RAPD markers, SNPs, and SSRs) that identify genetically linked markers which are proximate to the single nucleotide polymorphism. The method consisting of a homogeneous assay detection system for a PCR process using FRET allows for tracking of the single nucleotide polymorphism encoded herbicide-resistance trait in the progeny of a plant breeding cross. The method consisting of a homogeneous assay detection system for a PCR process using FRET of the present disclosure can be used to identify any sunflower variety containing the single nucleotide polymorphism.

Disclosed methods further comprise selecting progeny of said plant-breeding cross by analyzing said progeny for a single nucleotide polymorphism which is detectable according to the subject disclosure. For example, the method consisting of a homogeneous assay detection system for a PCR process using FRET can be used to track a single nucleotide polymorphism through breeding cycles with plants comprising other desirable traits, including both transgenic traits and non-transgenic traits. Plants comprising the single nucleotide polymorphism and the second desired trait can be detected, identified, selected, and quickly used in further rounds of breeding introgression by using the method of the subject disclosure. The single nucleotide polymorphism can also be combined through breeding introgression, and tracked or identified according to the subject disclosure, with a modified oil trait, an insect resistant trait(s), an agronomic trait, and/or with further herbicide tolerance traits. One preferred embodiment of the latter is a plant comprising the single nucleotide polymorphism combined with a gene encoding a modified oil trait.

The subject disclosure can be used in the determination of zygosity at one or more loci. Such a zygosity determination is useful in plant breeding introgression for the purpose of evaluating the level of inbreeding (that is, the degree of gene fixation), segregation distortion (i.e., in transgenic germplasm, maternal inheritance testing or for loci that affect the fitness of gametes), and the level of outbreeding (i.e., the relative proportion of homozygosity and heterozygosity). In determining zygosity, plants are identified which are homozygous or heterozygous (also described as hemizygous) at one or more loci. Any given plant can be homozygous or heterozygous for a given single nucleotide polymorphism, or homozygous or heterozygous for wildtype allele. The determination of zygosity at one or more loci can be used to estimate hybridity and whether a particular seed lot meets a commercial or regulatory standard for sale as certified hybrid seed. In addition, in transgenic germplasm, it is useful to know the ploidy, or copy number, to aid in trait integration strategies. In an preferred embodiment the method consisting of a homogeneous assay detection system for a PCR process using FRET is used to determine the zygosity of the an HaAHASL1 allele within sunflower plants.

Definitions and examples are provided herein to help describe the present disclosure and to guide those of ordinary skill in the art to practice the disclosure. Unless otherwise noted, terms are to be understood according to conventional usage by those of ordinary skill in the relevant art. The nomenclature for DNA bases as set forth at 37 CFR §1.822 is used.

Also, the indefinite articles “a” and “an” preceding an element or component of the disclosure are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The terms “nucleic acid,” “polynucleotide,” “polynucleotide sequence,” and “nucleotide sequence” are used to refer to a polymer of nucleotides (A,C,T,U,G, etc. or naturally occurring or artificial nucleotide analogues), e.g., DNA or RNA, or a representation thereof, e.g., a character string, etc, depending on the relevant context. The terms “nucleic acid” and “polynucleotide” are used interchangeably herein; these terms are used in reference to DNA, RNA, or other novel nucleic acid molecules of the disclosure, unless otherwise stated or clearly contradicted by context. A given polynucleotide or complementary polynucleotide can be determined from any specified nucleotide sequence. A nucleic acid may be in single- or double-stranded form.

The term “isolated” or “isolating” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material.

The term “plant,” includes plants and plant parts including but not limited to plant cells and plant tissues such as leaves, stems, roots, flowers, pollen, and seeds. The class of plants that can be used in the present disclosure is generally as broad as the class of higher and lower plants amenable to mutagenesis including angiosperms (monocotyledonous and dicotyledonous plants), gymnosperms, ferns and multicellular algae. As used herein, a “line” is a group of plants that display little or no genetic variation between individuals for at least one trait. Such lines may be created by several generations of self-pollination and selection, or vegetative propagation from a single parent using tissue or cell culture techniques.

The terms “cultivar” and “variety” are synonymous and refer to a line which is used for commercial production.

The term “amplification process” refers to any polymerase chain reaction based method which is used to amplify a polynucleotide fragment. Such methods utilize oligonucleotide primer sequences and DNA polymerases to synthesize a copy of complementary polynucleotide fragment through a series of thermal cycles the resulting amplicon is referred to as an “amplified product.”

The term “single nucleotide polymorphism” or “SNP” is a DNA sequence variation which occurs within the genome of an organism, wherein a single nucleotide base differs between members of a species. The DNA sequence variation usually results in a change in the single nucleotide base which is different from the expected nucleotide base at that position. The term “mutant allele” is used to refer to a change in the single nucleotide base from the sequence which is found in the majority of the species to an unexpected and different single nucleotide base not commonly found within the species. The term “wildtype allele” is used to refer to the presence of the expected single nucleotide base which is found in the majority of the species.

The term “zygosity” refers to the similarity of alleles of a gene for a trait (inherited characteristic) in an organism. If both alleles are the same, the organism is homozygous for the trait. If both alleles are different, the organism is heterozygous or hemizygous for that trait.

Embodiments of the present disclosure are further defined in the following Examples. It should be understood that these Examples are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### EXAMPLES

### Example 1

### DNA Extraction and Quantification

Plants from a sunflower line which were previously characterized as homozygous for the HaAHASL1-A122(At)T mutant allele were used to develop a method to detect the HaAHASL1-A122(At)T mutant allele. In addition, a second sunflower line which was previously characterized as homozygous for the HaAHASL1 wildtype allele was used to develop a method to detect the HaAHASL1 wildtype allele. Resultantly, an HaAHASL1 method consisting of a homogeneous assay detection system for a PCR process using FRET to detect and distinguish the mutant and wildtype alleles was developed. After the HaAHASL1 method consisting of a homogeneous assay detection system for a PCR process using FRET was developed, it was validated using sunflower lines that had been previously genotyped.

Genomic DNA was extracted from leaf tissue of the sunflower line homozygous for the HaAHASL1-A122(At)T mutant allele and the sunflower line homozygous for the HaAHASL1 wildtype allele using a QiaGene DNeasy 96 Plant Kit and quantified with PICOGREEN® dsDNA quantification kit (Life Technologies, Carlsbad, Calif.).

### Example 2

### Primer Design

Three primers (Table 1) were manually designed based on the nucleotide sequence information for the HaAHASL1 (SEQ ID NO:1) and the HaAHASL1-A122(At)T mutant allele (SEQ ID NO:2).

Mutant allele detection common primer 043-0001.1.A1 (SEQ ID NO:3) containing a tail on the 5′ end that is sequence identical to a KBiosciences reaction kit fluorescent-labeled primer was synthesized. The KBiosciences reaction kit fluorescent-labeled primer is labeled with the fluorescent dye, 5-carboxyfluorescein (FAM). This primer was specifically designed to amplify the HaAHASL1-A122(At)T mutant allele.

Wildtype allele detection common primer 043-0001.2.A2 (SEQ ID NO:4), containing a tail on the 5′ end that is identical to a KBiosciences reaction kit fluorescent-labeled primer was synthesized. The KBiosciences reaction kit fluorescent-labeled primer is labeled with the fluorescent dye, 2′,7′-dimethoxy-4′,5′-dichloro-6-carboxyfluorescein (JOE). This primer was designed to specifically amplify the HaAHASL1 wildtype allele.

Finally, a downstream common primer, 043.0001.8.0 (SEQ ID NO:5) which hybridizes to a sequence downstream of the HaAHASL1A122(At)T mutation site was used to amplify both the mutant and wildtype alleles.

All Primers were mixed to concentrations as listed in Table 2 in Tris-HCl (pH 8.3).

### Example 3

### HaAHASL1 Single Nucleotide Polymorphism Assay

The three HaAHASL1 common primers were mixed to achieve the concentrations described in Table 2. The HaAHASL1 reaction cocktail and thermocycling program are described in Tables 3 and 4, respectively. HaAHASL1 PCR reactions were performed in 96-well plate format on a GENEAMP® PCR System 9700 (Applied Biosystems, Carlsbad, Calif.). HaAHASL1 PCR reaction product results were read on a fluorescence plate reader using the following parameters: (1) FAM: excitation at ˜485 nm and emission at ˜535 nm; and (2) JOE: excitation at ˜525 nm and emission at ˜560 nm. Fluorescence reading data were saved in Microsoft Excel format and the data were plotted on an x-y axis. The FAM values were plotted on the X-axis and the JOE values on the Y-axis.

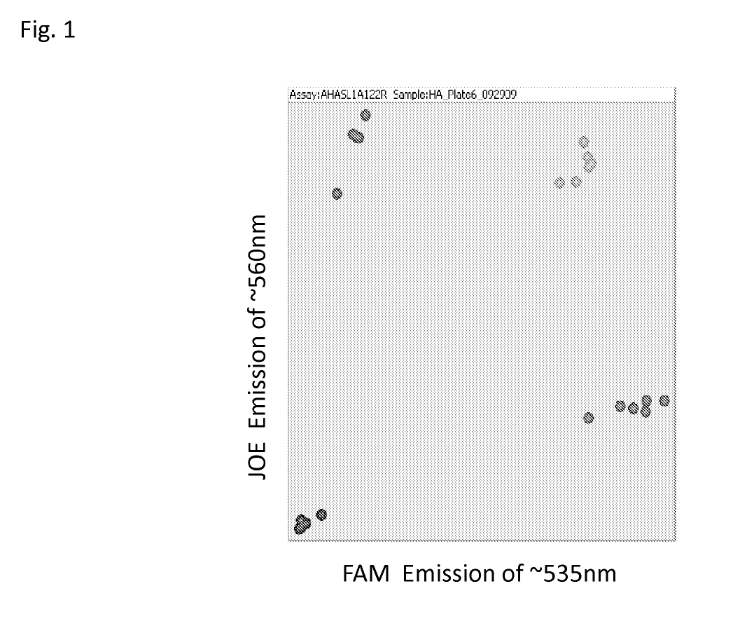

The results of the assay are presented in FIG. 1. The samples known to be homozygous for the HaAHASL1 wildtype allele clustered to the top left of the graph. The sunflower samples known to be homozygous for the HaAHASL1-A122(At)T mutant allele clustered in the bottom right of the graph. Plant samples that are hemizygous for the alleles cluster intermediately among these groups in the top right hand of the graph. The hemizygous samples were made by pooling DNAs from homozygous wildtype allele and homozygous mutant allele samples at an equal molar ratio. Finally, null experimental controls cluster at the lower left hand of the graph as a result of the background fluorescence readings. The null experimental controls were created by not including template DNA in the HaAHASL1 reactions. Each HaAHASL1 reaction was replicated six times for each genotype and experimental control sample.

The results demonstrated that the HaAHASL1 method could be used to identify the homozygous lines containing either the HaAHASL1-A122(At)T mutant allele or the HaAHASL1 wildtype allele. In addition, the hemizygous and the null control samples could be identified using the HaAHASL1 method.

TABLE 1
HaAHASL1 primer sequences.

| | Fluo- | SEQ | |
|---|---|---|---|
| | rescent | ID | |
| Primer ID | labeling | NO: | Sequence |
| 043- | FAM | SEQ | GAAGGTGACCAAGTTCATG |
| 0001.1.A1 | | ID | CTTGGTGGATCTCCATTGA |
| | | NO: 3 | CGT |
| 043- | JOE | SEQ | GAAGGTCGGAGTCAACGGA |
| 0001.2.A2 | | ID | TTCTTGGTGGATCTCCATT |
| | | NO: 4 | GACGC |
| 043.0001.8.0 | None | SEQ | AGACGTGTTGGTGGAAGCT |
| | | ID | CTG |
| | | NO: 5 | |

TABLE 2
Cocktail for 100 μl primer mixture.

| | Stock | | Working |
|---|---|---|---|
| | concentration | Volume | concentration |
| Allele Specific | 100 μM | 12 μl | 12 μM |
| Common Primer | | | |
| 043-0001.1.A1 | | | |
| Allele Specific | 100 μM | 12 μl | 12 μM |
| Common Primer | | | |
| 043-0001.2.A2 | | | |
| Downstream | 100 μM | 30 μl | 30 μM |
| Common Primer | | | |
| 043.0001.8.C | | | |
| Tris-HCl | 10 mM, pH 8.3 | 46 μl | — |

TABLE 3
Cocktail of HaAHASL1 reaction.

| Components | Volume | |
|---|---|---|
| DNA (5 ng/μl) | 4 | μl |
| 2X Reaction Mix (KBiosciences Ltd.) | 4 | μl |
| Primer Mix (Table 3) | 0.11 | μl |
| Total | 8.11 | μl |

TABLE 4
Thermocycling program for HaAHASL1 reaction.

| | No. of | | | | |
|---|---|---|---|---|---|
| Steps | Cycles | Condition | Temperature | Duration | |
| 1: Hot-start Taq | 1 | Denaturation | 94° C. | 15 | min |
| activation | | | | | |
| 2: High stringency | 20 | Denaturation | 94° C. | 10 | sec |
| amplification | | Annealing | 57° C. | 5 | sec |
| | | Elongation | 72° C. | 10 | sec |
| 3: Low stringency | 22 | Denaturation | 94° C. | 10 | sec |
| amplification | | Annealing | 57° C. | 20 | sec |
| | | Elongation | 72° C. | 40 | sec |

### Example 4

### HaAHASL1 Method Consisting of a Homogeneous Assay Detection System for a PCR Process Using FRET Validation

To validate the HaAHASL1 method consisting of a homogeneous assay detection system for a PCR process using FRET, sunflower plants which were produced from breeding crosses were genotyped using the assay described above. The homozygous wildtype sunflower lines, homozygous HaAHASL1-A122(At)T mutant allele sunflower lines, and the hemizgyous and null experimental controls were included in the assay. The results of the HaAHASL1 method consisting of a homogeneous assay detection system for a PCR process using FRET were compared to results generated from a gel based assay performed on the same sample set. The gel based assay PCR products were resolved on a 2% agarose TAE gel, gel images were captured using UV transillumination, and genotype data were collected and collated manually. Consistent results were observed between the HaAHASL1 method consisting of a homogeneous assay detection system for a PCR process using FRET and the standard gel-based assay. In some cases the HaAHASL1 method consisting of a homogeneous assay detection system for a PCR process using FRET produced better resolution than the gel-based assay, thereby eliminating ambiguous genotyping patterns. For example, the gel-based assay detected a questionable zygosity pattern in one hybrid whereas the HaAHASL1 method consisting of a homogeneous assay detection system for a PCR process using FRET clearly confirmed that this particular hybrid was heterozygous.

A method consisting of a homogeneous assay detection system for a PCR process using FRET for detection of the HaAHASL1-A122(At)T mutant allele in sunflower was developed and validated. This assay is high-throughput, cost-effective, and highly efficient. This new assay will significantly improve the capability and precision of detecting for the sunflower plants containing the imidazolinone tolerant HaAHASL1-A122(At)T mutant allele and increase breeding selection efficiency through marker assisted selection.

The previous examples describe a method consisting of a homogeneous assay detection system for a PCR process using FRET which was developed to isolate and identify Helianthus annuus plants which contain the HaAHASL1-A122(At)T single nucleotide polymorphism mutant allele. In addition, this method can be used to determine the zygosity of Helianthus annuus plant which contain the HaAHASL1-A122(At)T and for marker assisted selection or integration of the HaAHASL1-A122(At)T single nucleotide polymorphism mutant allele into progeny plants.

What is claimed is:

1. A method consisting of a homogeneous assay detection system for a PCR process using FRET for detecting the presence or absence of a HaAHASL1-A122(At)T SNP within the HaAHASL1 gene, comprising:
a. isolating a genomic DNA sample from Helianthus annuus;
b. adding a set of oligonucleotide primers to said isolated genomic DNA sample, wherein said set of oligonucleotide primers are comprised of a mutant allele detection common primer consisting of SEQ ID NO:3, a wildtype allele detection common primer consisting of SEQ ID NO:4, a downstream common primer consisting of SEQ ID NO:5, and fluorescent-labeled primers;
c. subjecting said isolated genomic DNA sample and said set of oligonucleotide primers to an amplification process; and,
d. detecting at least one amplified product, wherein the amplified product indicates the presence or absence of a HaAHASL1-A122(At)T SNP.

2. The method of claim 1, wherein said amplified product consists of 84 base pairs.

3. The mutant allele detection common primer of claim 1, wherein said mutant allele detection common primer contains a tail sequence which is identical in sequence to a first fluorescent-labeled primer.

4. The first fluorescent-labeled primer of claim 3, comprised of a fluorescent dye.

5. The fluorescent dye of claim 4, comprising a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, or a ROX fluorescent dye.

6. The wildtype allele detection common primer of claim 1, wherein said wildtype allele detection common primer contains a tail sequence which is identical in sequence to a second fluorescent-labeled primer.

7. The second fluorescent-labeled primer of claim 6, comprised of a fluorescent dye.

8. The fluorescent dye of claim 7, comprising a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, or a ROX fluorescent dye.

9. The method of claim 1, wherein said method is used to determine zygosity comprising:
a. quantitating said first fluorescent dye of the fluorescent-labeled primer which is identical in sequence to the tail of the mutant allele detection common primer;
b. quantitating said second fluorescent dye of the fluorescent-labeled primer which is identical in sequence to the tail of the wildtype allele detection common primer;
c. comparing amounts of first fluorescent dye to second fluorescent dye; and,
d. determining zygosity by comparing fluorescence ratios of first fluorescent dye to second fluorescent dye.

10. A method of any one of the preceding claims, wherein the site of said present or absent SNP is located between SEQ ID NO:3 and SEQ ID NO:5 of Helianthus annuus chromosome 9.

11. A method of any one of the preceding claims, wherein the site of said present or absent SNP is located between SEQ ID NO:4 and SEQ ID NO:5 of Helianthus annuus chromosome 9.

12. The method of claim 1, wherein said method is used for breeding introgression into a second line of Helianthus annuus.

13. The breeding introgression method of claim 12, wherein said second line of Helianthus annuus does not contain a HaAHASL1-A122(At)T allele.

14. The breeding introgression method of claim 12, wherein said method is used to detect the presence or absence of a HaAHASL1-A122(At)T SNP within the HaAHASL1 gene in progeny plants.

15. The method of claim 1, wherein said method is used to identify lines of Helianthus annuus that possesses imidazolinone herbicide tolerance.


SEQUENCE LISTING (5 sequences)

SEQ ID NO: 1 - DNA, 86 bp, Helianthus annuus
agacgtgttggtggaagctctggaacgggaaggtgtcaccgacgtcttcgcctaccccggcggcgcgtcaatggagatccaccaag

SEQ ID NO: 2 - DNA, 84 bp, Helianthus annuus
agacgtgttggtggaagctctggaacgggaaggtgtcaccgacgtcttcgcctaccccggcggcacgtcaatggagatccacca

SEQ ID NO: 3 - DNA, 41 bp, Artificial Sequence
  Mutant allele detection common primer, 043-0001.1.A1
gaaggtgaccaagttcatgcttggtggatctccattgacgt

SEQ ID NO: 4 - DNA, 43 bp, Artificial Sequence
  wild type allele detection common primer, 043-0001.2.A2
gaaggtcggagtcaacggattcttggtggatctccattgacgc

SEQ ID NO: 5 - DNA, 22 bp, Artificial Sequence
  downstream common primer, 043.0001.8.C
agacgtgttggtggaagctctg